(19) United States
(12) Patent Application Publication
(10) Pub. No.: US 2007/0179323 A1
(43) Pub. Date: Aug. 2, 2007

(54) 3(4),7(8)-Dihydroxymethylbicyclo[4,3,0]nonane and a process for its preparation

(75) Inventors: Helmut Springer, Dinslaken (DE); Paolo Bavaj, Frankfurt (DE)

Correspondence Address: HEDMAN & COSTIGAN P.C., 1185 AVENUE OF THE AMERICAS, NEW YORK, NY, 10036, US

(73) Assignee: Celanese Chemicals Europe GmbH

(21) Appl. No.: 11/657,771

(22) Filed: Jan. 25, 2007

(30) Foreign Application Priority Data
Jan. 31, 2006 (DE) 10 2006 004 318.9

(51) Int. Cl. C07C 35/20

(52) U.S. Cl. 568/821

(57) ABSTRACT

The novel chemical compound, 3(4),7(8)-dihydroxymethylbicyclo[4.3.0]nonane and a process for its preparation, wherein bicyclo[4.3.0]nona-3,7-diene is reacted with synthesis gas in homogeneous organic phase in the presence of transition metal compounds of Group VIII of the Periodic Table containing complex-bound organophosphorus compound, at 70 to 160° C. and pressures of 5 to 35 MPa, and the 3(4),7(8)-bisformylbicyclo[4.3.0]nonane thus obtained is hydrogenated.

The present invention relates to 3(4),7(8)-dihydroxymethylbicyclo [4.3.0]nonane and to a process for its preparation from bicyclo [4.3.0]nona-3,7-diene.

### STATE OF THE ART

Fused alicyclic unsaturated hydrocarbons with isolated double bonds in the rigns are valuable starting materials which can be converted to compounds with important uses. The cyclic and fused hydrocarbon skeleton imparts particular properties. One example of this compound class is dicyclopentadiene (DCP), which is readily available by dimerizing cyclopentadiene and is also prepared on an industrial scale, and can be converted to compounds with important uses, to which the tricyclodecane skeleton imparts particular properties. The compounds which are derived from DCP and have a tricyclodecane structure are frequently also referred to in the literature as TCD derivatives (Chemiker-Zeitung, 98, 1974, pages 70 to 76).

Especially the hydroformylation of DCP affords TCD aldehydes of interest, such as 3(4),8(9)-bisformyltricyclo[5.2. 1.0^{2,6}]decane, also known as TCD dialdehyde, which is processed further to give important intermediates. Owing to its thermal lability, which leads to losses in the course of distillative workup, TCD dialdehyde is usually not isolated in pure form but rather processed further as the crude product of the hydroformylationreaction. For instance, the hydrohenation of TCD dialdehyde leads to TCD alcohol DM {3(4),8(9)-dihydroxymethyltricyclo[5.2.1.0^{2,6}]decane}, which has great economic significance as a valuable intermediate for the chemical industry. The dihydric alcohol is of high industrial interest in various ways for different applications: acrylic esters and methacrylic esters of OH-containing tricyclic decanols (DE 2 200 021 A), as a constituent of acrylic ester adhesives which cure with exclusion of oxygen, (meth)acrylic esters of ether-containing tricyclic decanols (EP 23 686 A2), for the production of adhesives and sealants, esters and polyesters of the tricyclodecane series (DE 934 889 C), which are suitable as plasticizers and high-value ester lubricants, odorant compositions (DE 2 307 627 A1) and acid sterilization-resistant polyester coatings (DE 3 134 640 C1) in the metal paint systems sector.

The preparation of aldehydes by catalytic addition of carbon monoxide and hydrogen to olefinic double bonds is known. While this reaction used to be performed virtually exclusively with cobalt as the catalyst, modern processes work with metallic rhodium or with rhodium compounds as catalysts, which are used alone or with complexing ligaments, for example organophosphines or esters of phosphorous acid. According to the unanimous opinion in the technical field, catalysts effective under the reaction conditions are hydrido carbonyl compounds of rhodium which can be reproduced by the general formula H[Rh(CO)₄₋ₓLₓ] where L is a ligand and x is 0 or an integer from 1 to 3.

A special case is the hydroformylation of dienes. While almost exclusively monoaldehydes are obtained under the customary conditions of the oxo process in the hydroformylation of conjugated dienes, it is possible to obtain not only the mono- but also the disubstitution products from dicyclopentadiene (DCP) with its isolated double bonds. Owing to the great significance of the hydroformylation products of DCP, there are also numerous studies in the technical literature which address both the hydroformylation reaction of DCP and the subsequent workup of the crude product. For instance, DE 38 22 038 A1 and GB 1 170 226 consider the hydroformylation of DCP in the presence of rhodium in an organic solvent at elevated pressure and elevated temperature. A comprehensive review of the hydroformylationof dicyclopentadiene can be found in the Chemiker-Zeitung 98, 1974, 70-76, where reference is likewise made to the thermal lability of the TCD aldehydes, which leads to high product losses in the distillative workup of the crude hydroformylation mixture. Therefore, TCD dialdehydes are usually not isolated in pure form but processed further in their mixtures with the by-products of the oxo process. However, indications of extractive workup processes without thermal stress can also be found in the prior art, for example in EP01 065 194 A1 or U.S. Pat. No.5,138,101 A. In these processes, the organic crude mixture is extracted with a polar organic solvent, for example with a polyhydric alcohol or with a methanol/water mixture, which transfers the TCD dialdehydes to the polar alcoholic phase, and the hydroformylation catalyst remains in the hydrocarbon phase.

As a consequence of the various possible uses, TCD alcohol DM is of high economic interest and the patent literature contains numerous references to processes for its preparation.

U.S. Pat. No. 4,647,708 A describes the hydroformylation of dicyclopentadiene using Rh as a catalyst in the presence of ion exchangers (Dowex® MWA-1) in toluene/THF as solvents. The reaction is effected at 120° C. and 27.5 MPa of CO/H₂ (in a ratio of 1:2) in two separate continuous autoclaves. With reference to the experimental results disclosed, it can be seen that the yield of TCD alcohol DM declines from 85% to 65% within the 30-day experimental period. The reaction system is thus unsuitable for industrial use.

U.S. Pat. No. 4,262,147 A describes the use of bimetallic Rh/Co clusters on resins such as Amberlite® IRA-68. Under the conditions employed (110° C., 11 MPa, 8 hours), a selectivity of 68% of TCD alcohol DM is obtained in this one-stage synthesis.

Owing to the great economic significance that diols based on fused, alicyclic hydrocarbons have, there is therefore a need for the provision of further, inexpensively available diols in high purity, which have a cyclic hydrocarbon skeleton with fused rings.

### OBJECTS OF THE INVENTION

It is an object of the invention to provide the novel compound, 3(4),7(8)-dihydroxymethylbicyclo[4.3.0]nonane.

It is another object of the invention to provide a process for the preparation of the said compound starting from bicyclo[4.3.0]nona-3,7-diene.

These and other objects and advantages of the invention will become obvious from the following detailed description.

### THE INVENTION

The invention comprises a process for preparing 3(4),7(8)dihydroxymethyl-bicyclo[4.3.0]nonane comprising hydroformylating bicyclo[4.3.0]nona-3,7-diene followed by subsequent hydrogenation. It comprises reacting bicyclo[4.3.0]nona-3,7-diene with synthesis gas in homogeneous organic phase in the presence of transition metal compounds of Group VIII of the Periodic Table containing complex-bound organophosphorus compounds, and of excess organophosphorus compound, at temperatures of 70 to 160° C. and pressures of 5 to 35 MPa, and then hydrogenating the 3(4),7(8)-bisformyl-bicyclo[4.3.0]nonane thus obtained to 3(4),7(8)-dihydroxymethyl-bicyclo[4.3.0]nonane.

The inventive compound derives from bicyclo[4.3.0]nona-3,7-diene, which is prepared industrially by Diels-Alder reaction of butadiene with cyclopentadiene and which is therefore available in inexpensive amounts.

The numbering of the carbon atoms bonded in the unsaturated, bicyclic hydrocarbon is according to the following sequence:

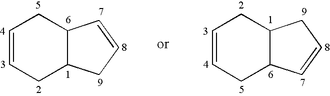

the two structural formulae being identical.

The inventive compound 3(4),7(8)dihydroxymethylbicyclo[4.3.0]nonane is a mixture of different isomers of dihydroxymethylbicyclo[4.3.0]nonane in which the hydroxymethyl group in the six-membered ring can be bonded once at the 3- or at the 4-position, and the hydroxymethyl group in the five membered ring once at the 7- or at the 8-position.

In analogy to the notation customary for the TCD derivatives according to Chemiker-Zeitung, 98, 1974 pages 70 to 76, the inventive compound 3(4),7(8)-dihydroxymethylbicyclo[4.3.0]nonane can be described in terms of formula as follows:

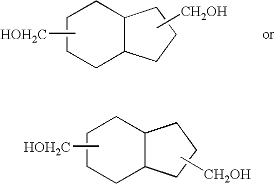

the two structural formulae being identical.

The starting material, bicyclo[4.3.0]nona-3,7diene, can be subjected to the hydroformylation as such or in solution. Suitable solvents are those in which starting material, reaction product and catalyst are soluble and which behave inertly under the reaction conditions. Examples are water-insoluble ketones, dialkyl ethers, aliphatic nitrites, aromatic hydrocarbons such as benzene, toluene, the isomeric xylenes or mesitylene, and saturated cycloaliphatic hydrocarbons such as cyclopentane or cyclohexane, or saturated aliphatic hydrocarbons such as n-hexane, n-heptane or n-octane. The proportion of the solvent in the reaction medium can be varied over a wide range and is typically from 10 to 80% by weight, preferably from 20 to 50% by weight, based on the reaction mixture.

The hydroformylation stage is performed in a homogeneous reaction system. The term homogeneous reaction system means a homogeneous solution composed essentially of solvent, if added in the reaction stage, catalyst, excess organophosphorus compound, unconverted starting compound and hydroformylation product.

The catalysts used are transition metal compounds of Group VIII of the Periodic Table which contain complex-bound organophosphorus compounds. Preference is given to using complexes of cobalt, rhodium, iridium, nickel, iron, platinum, palladium or ruthenium, and preferred are cobalt, rhodium and iridium. Particular preference is given to the use of rhodium complexes which contain organic phosphorus (III) compounds as ligands. Such complexes and their preparation are known (for example from U.S. Pat. No.3,527,809 A, U.S. Pat. No. 4,148,830 A, U.S. Pat. No. 4,247,486 A and U.S. Pat. No. 4,283,562 A). They may be used as homogeneous complexes or as a mixture of different complexes. The rhodium concentration in the reaction medium extends over a range of from 5 to 1000 ppm by weight and is preferably from 10 to 700 ppm by weight. In particular, rhodium is employed in concentrations of from 20 to 500 ppm by weight, based in each case on the homogeneous reaction mixture.

The hydroformylation is performed in the presence of a catalyst system composed of rhodium-organophosphorus complex and free, i.e. excess, organophosphorus ligand which does not enter into a complex with rhodium. The free organophosphorus ligand may be the same as in the rhodium complex, but it is also possible to use different ligands. The free ligand may be a homogeneous compound or consist of a mixture of different organophosphorus compounds. Examples of rhodium-organophosphorus complexes which may find use as catalysts are described in U.S. Pat. No. 3,527,809 A.

The preferred ligands in the rhodium complex catalysts include, for example, triarylphosphines such as triphenylphosphine, trialkylphosphines such as tri(n-octyl)phosphine, trilaurylphosphine, tri(cyclohexyl)phosphine, alkylarylphosphines, alkyl phosphites, aryl phosphites, alkyl diphosphites and aryl diphosphites. For instance, it is likewise possible to use rhodium complexes which contain aryl phosphites of the formula P(OR¹)(OR²)(OR³) in complex-bound form, wherein at least one of R¹, R² or R³ is an ortho-substituted phenyl. Suitable complex ligands have been found to be tris(2-tert-butylphenyl)phosphite or tris(2-tert-butyl4-methylphenyl)phosphite. The rhodium-catalyzed hydroformylation of olefins with phosphite-modified complexes is known from EP 0 054 986 A1. Owing to its easy availability, triphenylphosphine is employed particularly frequently.

Typically, the molar ratio of rhodium to phosphorus in the homogeneous reaction mixture is from 1:5 to 1:200, but the molar proportion of the phosphorus in the form of organic phosphorus compounds may also be higher. Preference is given to using rhodium and organically bound phosphorous in molar ratios of from 1:10 to 1:100.

When a transition metal of Group VIII of the Periodic Table other than rhodium is used in the hydroformylation stage, the concentration of transition metal and the molar ratio of transition metal to phosphorus is within the ranges which are also selected for rhodium. The optimal values in each case can be determined by simple routine tests depending on the transition metal used in each case.

The conditions under which the hydroformylation proceeds can vary within wide limits and can be adjusted to the individual circumstances. They depend upon factors including the starting material, the catalyst system used and the desired conversion. Typically, the hydroformylation of bicyclo[4.3.0]nona-3,7-diene is performed at temperatures of from 70 to 160° C. Preference is given to maintaining temperatures of from 80 to 150° C. and in particular from 90 to 140° C. The total pressure extends over a range of from 5 to 35 MPa, preferably from 10 to 30 MPa and in particular from 20 to 30 MPa. The molar ratio of hydrogen to carbon monoxide varies typically between 1:10 and 10:1; mixtures which contain hydrogen and carbon monoxide in a molar ratio of from 3:1 to 1:3, especially about 1:1, are particularly suitable.

The catalyst is typically formed from the transition metal or transition metal compound, organophosphorus compound and synthesis gas under the conditions of the hydroformylation reaction in the reaction mixture. However, it is also possible first to preform the catalyst and then to feed it to the actual hydroformylation stage. The conditions of the preformation correspond generally to the hydroformylation conditions.

For the preparation of the hydroformylation catalyst, the transition metal of Group VIII of the Periodic Table, especially rhodium, is used either in metallic form or as a compound. In metallic form, the transition metal is used either in the form of fine particles or in a thin layer on a support, such as activated carbon, calcium carbonate, aluminium silicate, alumina. Suitable transition metal compounds are salts of aliphatic mono- and polycarboxylic acids such as transition metal 2-ethylhexanoates, acetates, oxalates, propionates or malonates. In addition, it is possible to use salts of inorganic hydrogen and oxygen acids such as nitrates or sulfates, the various transition metal oxides or else transition metal carbonyl compounds such as Rh₃(CO)₁₂, Rh₆(CO)₁₆, CO₂(CO)₈, CO₄(CO)₁₆, Fe(CO)₅, Fe₂(CO)₉, Ir₂(CO)₈, Ir₄(CO)₁₂ or transition metal complexes, for example cyclopentadienylrhodium compounds, rhodium acetylacetonate, cyclopentadienylcobalt(cyclooctodiene-1,5), Fe(CO)₃(cyclooctadiene-1,5), [RhCl(cyclooctadiene-1,5]₂ or PtCl₂(cyclooctadiene-1,5). Transition metal-halogen compounds are less useful owing to their corrosive behavior of the halide ions.

Preference is given to transition metal oxides and, in particular, transition metal acetates and 2-ethylhexanoates. Particular suitable compounds have been found to be rhodium oxide, rhodium acetate, rhodium 2-ethyl-hexanoate, cobalt oxide, cobalt acetate and cobalt 2-ethylhexanoate.

The hydroformylation stage may be performed either batchwise or continuously. In the process of the invention, the starting olefin bicyclo[4.3.0]nona-3,7-diene is converted virtually completely, and a crude hydroformylation product having a content of the desired bisformyl product which is generally above 75% by weight based on the crude hydroformylation product is obtained.

The reaction product of the hydroformylation stage is supplied to the hydrogenation stage without further purification and without catalyst removal. The hydrogenation of the crude 3(4),7(8)bisformylbicyclo[4.3.0]nonane to give 3(4),7(8)-dihydroxymethylbicyclo[4.3.0]nonane is effected under generally customary reaction conditions in the presence of conventional hydrogenation catalysts. In general, the hydrogenation temperature is from 70 to 170° C. and the pressure employed is from 1 to 30 MPa. Suitable hydrogenation catalysts are particularly nickel catalysts.

The catalytically active metal can be applied on a support, generally in an amount of from about 5 to about 70% by weight, preferably from about 10 to 65% by weight and in particular from about 20 to about 60% by weight, based in each case on the total weight of the catalyst. Suitable catalyst supports are all conventional support materials, for example aluminum oxide, aluminum oxide hydrates in their various manifestations, silicon dioxide, polysilicic acids (silica gels) including kieselguhr, silica xerogels, magnesium oxide, zinc oxide, zirconium oxide and activated carbon. In addition to the main components of nickel and support material, the catalysts may also comprise additives in minor amounts, which serve, for example, to improve their hydrogenation activity and/or their lifetime and/or their selectivity. Such additives are known; they include, for example, the oxides of sodium, potassium, magnesium, calcium, barium, zinc, aluminum zirconium and chromium. They are added to the catalyst generally in a total proportion of from 0.1 to 50 parts by weight based on 100 parts by weight of nickel.

However, it is also possible to use unsupported catalysts such as Raney nickel or Raney cobalt in the hydrogenation process.

The hydrogenation stage is performed batchwise or continuously in the liquid phase with suspended catalysts or in the liquid or gaseous phase with fixed bed catalysts; the continuous procedure is preferred.

In a batchwise process, based on 3(4),7(8)-bisformylbicyclo[4.3.0]nonane, from 1 to 10% by weight, preferably from 2 to 6% by weight, of nickel is used in the form of the above-described catalysts. In a continuous method, from about 0.05 to about 5.0 kg of the 3(4),7(8)-bisformylbicyclo[4.3.0]nonane are used per liter of catalyst and hour; preference is given to using from about 0.1 to 2.0 kg of 3(4),7(8)-bisformylbicyclo[4.3.0]nonane per liter of catalyst and hour.

The hydrogenation is effected preferably with pure hydrogen. However, it is also possible to use mixtures which comprise free hydrogen and additionally constituents which are inert under the hydrogenation conditions. In any case, it should be ensured that the hydrogenation gas is free of catalyst poisons such as sulfur compounds or carbon monoxide in harmful amounts.

Crude 3(4),7(8)-bisformylbicyclo[4.3.0]nonane can be used as such or together with a solvent or diluent, the latter variant being preferred owing to the high viscosity of the diol formed. When a solvent or diluent is added, the selection of the solvents or diluents, which may be pure substances or else substance mixtures, is not critical provided that it is ensured that they form a homogeneous solution with the feedstock and the reaction product. Examples of suitable solvents or diluents are linear or cyclic ethers such as tetrahydrofuran or dioxane, and also aliphatic alcohols, for example methanol, ethanol, butanol and isobutanol. The amount of the solvent or diluent used may be selected freely according to the apparatus and process technology circumstances; in general, solutions are used which contain from 10% to 75% by weight of 3(4),7(8)-bisformylbicyclo[4.3.0]nonane.

The pure 3(4),7(8)dihydroxymethylbicyclo[4.3.0]nonane is obtained by conventional distillation processes. The cyclic diol is drawn off as the top product. Residue amounts of the transition metal used in the hydroformylation stage are obtained in the distillation residue and are recovered by known processes.

The reaction product of the hydroformylation of bicyclo[4.3.0]nona-3,7-diene can also first be distilled by conventional processes and hydrogenated as a purified product. Surprisingly, 3(4),7(8)-bisformylbicyclo[4.3.0]nonane can be obtained in pure form with high distillative yield. This is all the more surprisingly because the prior art points out the thermal lability of dialdehydes with fused alicyclic ring structures. Transition metal, preferably rhodium, and added organophosphorus compounds are obtained in the distillation residue and are recovered by known methods. The subsequent hydrogenation of the purified 3(4),7(8)-bisformylbicyclo[4.3.0]nonane is effected as in the case of the reaction of the crude hydroformylation product.

The process of the invention permits a simple and inexpensive route to 3(4),7(8)-dihydroxymethylbicyclo[4.3.0]nonane in high yield and in high purity. The diol prepared by the process according to the invention can be used in an excellent manner for different application, for examples as a constituent in polyurethanes, polyesters or acrylic esters, and for the preparation of conversion products which are used as plasticizers or lubricants.

In the following examples, there are described several preferred embodiments to illustrate the invention. However, it should be understood that the invention is not intended to be limited to the specific embodiments.

### EXAMPLES

### Preparation of 3(4),7(8)-dihydromethylbicyclo[4.3.0]nonane

### 1. Preparation of 3(4),7(8)-bisformylbicyclo[4.3.0]nonane

A steel autoclave with a magnetic stirrer was initially charged with 1000 g of bicyclo[4.3.0]nona-3,7-diene of technical quality and 1000 g of toluene. After adding 12.75 g of triphenylphosphine and 50 mg of rhodium in the form of a toluenic solution of rhodium 2-ethylhexanoate having a content of 7062 mg of Rh/kg, the mixture was heated to 130° C. and treated with synthesis gas under a pressure of 26 MPa. After a reaction time of 8 hours, the hydroformylation reaction was ended. The organic phase was analyzed by gas chromatography.

GC analysis (area percent without toluene)

| | |
|---|---|
| components in first runnings | 0.2 |
| bicyclo[4.3.0]nona-3,7-diene range | 0.1 |
| components | 4.6 |
| 3(4),7(8)-bisformylbicyclo[4.3.0]nonane | 89.1 |
| triphenylphosphine/triphenylphosphine oxide | 1.2 |
| high boilers | 4.8 |

### 2. Preparation of 3(4),7(8)-dihydromethylbicyclo[4.3.0]nonane

The crude 3(4),7(8)-bisformylbicyclo[4.3.0]nonane obtained after the hydroformylation was freed largely of the toluene by distillation on a thin-film evaporator (jacket temperature 140° C., pressure 100 hPa). A residue was obtained which, by gas chromatography analysis, also contained 9.5% other components in addition to 6.7% toluene and 83.8% 3(4),7(8)-bisformylbicyclo[4.3.0]nonane. Subsequently, 700 g of the residue diluted with 300 g of isobutanol, and 42 g of Ni 52/35 catalyst from Johnson Matthey Plc, were initially charged in a 3 liter autoclave. The reaction mixture was heated to 130° C. and reacted at a pressure of 10.0 MPa and a reaction time of 8 hours. After the reaction had ended, the reaction mixture was cooled, decompressed and filtered from the catalyst to obtain the reaction product which was analyzed by gas chromatography.

GC analysis (in area percent)

| | |
|---|---|
| components in first runnings | 1.3% |
| isobutanol/toluene/methylcyclohexane | 29.2% |
| 3(4),7(8)-dihydroxymethylbicyclo[4.3.0]nonane | 62.8% |
| others | 6.7% |

For workup, the crude hydrogenation product was distilled using a Claisen distillation system starting from 825.3 g, 459.3 g of main fraction in a boiling range of 178-179° C. were obtained at a pressure of 1 hPa with the following composition:

GC analysis (in area percent)

| | |
|---|---|
| components in first runnings | 0.1% |
| 3(4),7(8)-dihydroxymethylbicyclo[4.3.0]nonane | 97.3% |
| others | 2.6% |

The overall yield of 3(4),7(8)-dihydroxymethylbicyclo[4.3.0]nonane over all stages is 79.6% of theory based on bicyclo[4.3.0]nona-3,7-diene used.

### Characterization:

Elemental analysis

| | | |
|---|---|---|
| C₁₁H₂₀O₂ (184.3) | Calc. | C 71.7%, H 10.9%, O 17.3% |
| | Found. | C 70.6%, H 10.5%, O 16.7% |

### NMR data

¹H NMR (500 MHz, DMSO-d₆, ppm):0.60-2.20 (m, 14 H, CH and CH₂),
3.18-3.43 (m, 4H, CH₂O), 4.32 (s, 2 H, OH)
¹³C NMR (125 MHz, DMSO-d₆, ppm):23.46-48.82 (CH and CH₂),
62.39-66.80 (CH₂OH)

IR data (diamond ATR-IR spectroscopy)
ν (cm⁻¹) 3306 (m, br), 2911 (s), 2855 (s), 1445 (w), 1030 (s)

| Density at 60° C. | 1.051 g/cm³ |
|---|---|
| Refractive index n_{D} at 60° C. | 1.5000 |

The process of the invention opens up an elegant preparative route for 3(4),7(8)-dihydroxymethylbicyclo[4.3.0]nonane in high yields. The novel compound, 3(4),7(8)-dihydroxymethylbicyclo[4.3.0]nonane, has an alicyclic ring structure with fused rings, which is outstandingly suitable as a constituent for polyurethanes, polyesters or acrylic esters. It can likewise be used for the preparation of conversion products which are used as plasticizers and lubricants.

Various modifications of the process and product of the invention may be made without departing from the spirit or scope thereof and it is to be understood that the invention is intended to be limited as defined in the appended claims.

1. A process for the preparation of 3(4),7(8)dihydroxymethylbicyclo[4.3.0]nonane comprising hydroformylating bicyclo[4.3.0]nona-3,7-diene with subsequent hydrogenation, which comprises reacting bicyclo[4.3.0]nona-3,7-diene with synthesis gas in a homogeneous organic phase in the presence of transition metal compounds of Group VIII of the Periodic Table containing complex-bound organophosphorus compounds, and excess organophosphorus compound, at 70 to 160° C. and pressures of 5 to 35 MPa, and then hydrogenating the 3(4),7(8)-bisformylbicyclo[4.3.0]nonane thus obtained to form 3(4),7(8)-dihydromethylbicyclo[4.3 .0]nonane.

2. The process of claim 1, wherein the organophosphorus compounds are organic phosphorus (III) compounds selected from the group consisting of triarylphosphines, trialkylphosphines, alkylarylphosphines, alkyl phosphites, aryl phosphites, alkyl diphosphites and aryl diphosphites.

3. The process of claim 2, wherein the triarylphosphine used is triphenylphosphine and the aryl phosphite used is tris(2-tert-butylphenyl) phosphite or tris(2-tert-butyl-4-methylphenyl)phosphite.

4. The process of claim 1, wherein the transition metal compounds of Group VIII are compounds of a metal selected from the group consisting of rhodium, cobalt, iridium, nickel, palladium, platinum, iron and ruthenium.

5. The process of claim 1, wherein the transition metal compounds are compounds of rhodium.

6. The process of claim 5, wherein rhodium is used in a concentration of 5 to 1000 ppm by weight based on the homogeneous reaction mixture.

7. The process of claim 6, wherein rhodium is used in a concentration of 10 to 700 ppm by weight, based on the homogeneous reaction mixture.

8. The process of claim 1, wherein the molar ratio of rhodium to phosphorus is 1:5 to 1.200.

9. The process of claim 8, wherein the molar ratio of rhodium to phosphorus is 1:10 to 1:100.

10. The process of claim 1, wherein the hydroformylation is performed at 80 to 150° C. and at pressures of 10 to 30 MPa.

11. The process of claim 1, wherein the hydrogenation is performed in the presence of nickel catalysts at 70 to 170° C. and at pressures of from 1 to 30 MPa.

12. The process of claim 10 wherein the temperature is 90 to 140° C. and the pressure is 20 to 30 MPa.

13. The process of claim 7 wherein the rhodium concentration is 20 to 500 ppm by weight.

14. 3(4),7(8)-dihydroxymethyl-bicyclo[4.3.0]nonane.
